(19) United States
(12) Patent Application Publication
(10) Pub. No.: US 2007/0213403 A1
(43) Pub. Date: Sep. 13, 2007

(54) TAMOXIFEN RESPONSE IN PRE-AND POSTMENOPAUSAL BREAST CANCER PATIENTS

(76) Inventors: Goran Landberg, Limhamn (SE); Lisa Ryden, Angelholm (SE)

Correspondence Address: GAUTHIER & CONNORS, LLP, 225 FRANKLIN STREET, SUITE 2300, BOSTON, MA, 02110, US

(21) Appl. No.: 11/620,275

(22) Filed: Jan. 5, 2007

Related U.S. Application Data
(63) Continuation of application No. PCT/SE05/01116, filed on Jul. 6, 2005.

(30) Foreign Application Priority Data
Jul. 7, 2004 (SE) 0401790-1

(51) Int. Cl. A61K 31/135

(52) U.S. Cl. 514/646

(57) ABSTRACT

The present invention relates to the use of an inhibitor of the VEGFR2 receptor in the preparation of a pharmaceutical preparation for improving tamoxifen treatment response in pre- and postmenopausal breast cancer patients being estrogen receptor positive.

### TECHNICAL FIELD

The present invention relates to improvements in tamoxifen treatments after breast cancer treatments.

### BACKGROUND OF THE INVENTION

Adjuvant tamoxifen treatment for five years reduces recurrence by 50% and death by 28% in hormone receptor positive breast cancer (1,2). The beneficial effect of tamoxifen is restricted to hormone receptor positive patients with expression of estrogen receptor (ER) and/or progesterone receptor (PR) (1). Tamoxifen blocks one of two activating domains for ER in the estrogen response elements of the ER genes in breast epithelium (2). ER activated gene expression is efficiently inhibited by tamoxifen in the breast epithelium, where this activating domain is the most important. In the uterus, tamoxifen acts as an agonist since the other activating domain is predominant. Resistance to endocrine therapy with tamoxifen is a clinical problem and additional predictive markers besides hormone receptor contents are therefore being evaluated (11). Growth factors can activate ER without ligand-binding by growth-factor receptor tyrosine-kinases activation via different intracellular pathways (25, 26, 28). C-erbB2, also known as HER2, and EGFR act via direct interaction with phosphorylation of ER and overexpression of HER2 is associated with resistance to tamoxifen in in vitro experiments (5). Clinical data implicate that overexpression of HER2 may deteriorate the beneficial effect of tamoxifen, although evidence from randomised studies is still limited (7,12,30). Furthermore, the development of a monoclonal antibody, trastuzumab, directed towards the HER2 offers possibility to give specific therapy to patients that overexpress HER 2.

Vascular endothelial growth factor (VEGF) is the main pro-angiogenic growth factor and is a prognostic indicator of clinical outcome when measured in tumour tissue of primary breast cancer by ELISA methods (14, 19). The role of VEGF as a predictor of response to tamoxifen therapy in both early and advanced breast cancer has been addressed in non-randomised trials, preliminary indicating that high levels of cytosolic VEGF in hormone-responsive disease may deteriorate response to endocrine treatment (7, 10, 12, 14, 19, 22). The cross-talk between sex steroid hormones and VEGF is complex. The VEGF gene has an estrogen responding element identified and both estrogen and progesterone are known to up regulate expression and secretion of VEGF (16, 17, 18) and when tamoxifen acts as an estrogen agonist in in vitro studies, stimulating VEGF expression possibly contradicting the effect of tamoxifen (27). The secretion of VEGF to the extracellular compartment is reported to be inhibited by tamoxifen (13), although this cannot explain the association between high tissue levels of VEGF at time of diagnosis and impaired prognosis of tamoxifen treated patients. VEGF exhibits its effect via two tyrosine kinases receptors, VEGFR1 and VEGFR2, with an intracellular pathway similar to other tyrosine kinases like HER2 and EGFR, and are mainly located on endothelial cells, but have also been identified on tumour cells in breast cancer, implicating autocrine and paracrine roles for VEGF besides its pro-angiogenic property (3,29,32). Experimental data preliminary indicate that VEGF stimulates proliferation in estrogen-responsive breast cancer cell lines expressing VEGF receptors (15, 24), although conflicting result exist (29).

The aim of this study was to study the association of tumour specific expression of VEGF-A, VEGFR2 and HER 2 to tamoxifen response in two controlled, randomised trials of tamoxifen vs control including pre- and postmenopausal patients. In the first study including only premenopausal patients the beneficial effect by two years of tamoxifen in estrogen receptor positive disease on recurrence-free survival (RFS) was significant giving excellent opportunities to further explore additional predictive markers.

Patients and Methods

Study Design

1. Premenopausal Patients

Premenopausal patients or patients under 50 years with stage II (pT2 N0 M0, pT1 N1 M0 and pT2, N1 M0) invasive breast cancer were enrolled in a clinical trial between 1986 -1991 and randomized to control (n=288) or two years of tamoxifen (n=276). All patients were radically operated by modified radical mastectomy or breast conserving surgery with axillary lymph node dissection (level one and two). After breast conserving surgery, radiotherapy (50 Gy) was given to the breast and in patients with axillary lymph node metastases locoregional radiotherapy was delivered. Patients were included irrespective of hormone receptor status. Randomization was performed by the Regional Oncologic Centre and oral informed consent registered for all patients. The study was approved by the ethical committee at Lund and Linkoping Universities.

The patients were followed until 5 years with annual mammogram and physical examination and then within the national program by screening mammogram every 18^{th} month.The median duration of follow-up for patients without breast cancer event was 13.9 years (95% Cl: 13.6-14.3). The median follow-up time was the same in the two treatment arms.
2. Postmenopausal Patients

The patients included were enrolled in a multi-center clinical trial at Umea University Hospital, Sweden, during 1980-1987 (Trial II) included in the Oxford meta-analysis. The inclusion criteria were postmenopausal patients (>55 years) with stage II (pT1, pN1, pM0, pT2, pN0, pM0, pT2, pN2, pM0) invasive breast cancer. For patients older than 70 years, stage II and stage III breast cancer patients were included. Patients were operated with modified radical mastecomy. The hormone receptor level was not determined at time of randomization. Patients were randomized to control or tamoxifen treatment (40 mg/day) for two years by the Regional Oncologic Center and oral informed consent was registered on admission to the Oncological Clinic. The study was approved by the Ethical Committee at the Umea university 1980 as well as 2002 (modification regarding molecular markers used in tissue micro array). The median age was 66.5 years (55-75 years) at inclusion. The median follow-up time was 18 years (range 15-22 years) for surviving patients.

Tumour Tissue Array Preparation

Formalin-fixed, paraffin-embedded tumour blocks and clearly defined areas of tumour samples were arranged in a tissue micro array (TMA). Two biopsies, 0.6 mm, were obtained from each donor block, corresponding to a previously marked area on a slide of invasive tumour, and mounted in a recipient block using a tissue array machine according to the manufacturer's instructions (Beecher Instruments, MD, USA). The TMA was monitored after haematoxylin staining and in case of lack of visible areas of invasive tumour cells a duplicate biopsy was processed in a second round.

Immunohistochemistry

VEGF-A and VEGFR2

3-4 um sections of the paraffin embedded tissue arrays were dried, deparaffinized, rehydrated and microwave treated for 5+5 min in a citrate buffer (pH 6.0) before being processed in an automatic immunohistochemistry staining machine according to standard procedures (TechMate500, Dako, Denmark) using a polyclonal VEGF (A-20) antibody diluted 1:400 recognizing VEGF 165, 189 and 121 (Santa Cruz, Calif., USA) and a monoclonal VEGFR2 antibody recognizing VEGFR2/KDR diluted 1:1000 (Santa Cruz, Calif., USA). Normal human kidneys were used as positive controls for VEGF-A and for VEGFR2, human aortic endothelium served as positive controls. VEGF-A was previously validated using an ELISA-based method (LR). The cytoplasmatic staining intensity was evaluated semi-quantitatively using a classification from 0-3, representing lack of staining (0), low staining intensity (1), intermediate staining intensity (2) and high staining intensity (3). Only invasive tumor cells were evaluated. In survival analyses, high staining intensity (grade 3) was compared with the other grades (0, 1, 2) for both VEGF-A and VEGFR2 in premenopausal patients. In postmenopausal patients lack of staining was compared with staining of any intensity.

The TMA was examined by two investigators with blinded clinical data and the concordance between the investigators was 89% for VEGF-A and 93% for VEGFR2. Divergent results were re-examined followed by a conclusive decision.

Estrogen and Progesterone

ER and PR were determined by immunohistochemistry in a tissue microarray. For this purpose, the Ventana Benchmark system (Ventana Medical Systems Inc., AZ, USA) with prediluted antibodies (Anti-ER Clone 6F11 and Anti-PgR Clone 16) was used. In line with the clinically established cut-off used for hormone receptor assessment, tumors with more than 10% positively staining nuclei were considered positive. In addition, the fractions of positive nuclei were quantified.

HER2

Expression of HER2 was determined by immunhistochemistry in TMA and 420 of 500 tumor blocks were evaluable among the premenopausal patients. The Ventana antibody-kit was used with prediluted antibodies and tumors were evaluated semi-quantitatively. According to a protocol the tumors were scored as lack of staining (grade 0), weak staining intensity in tumor cell membranes (grade 1), intermediate staining intensity in tumor cell membranes in most of the tumor cells (grade 2) and intense staining intensity in all tumor cells (grade 3). The distribution is given in Table 1. The proportion of overexpression of HER2 (grade 3) was 15.2%. In 116 tumours, data was available for both erbB2-amplification and HER2. The association between HER2 overexpression (staining intensity 3) and erbB2 amplification was highly significant, p<0.001, with a kappa-value of 0.81.

Table 1

Comparison of patients and tumour characteristics of the original study and the present study in addition to markers analyzed by immunohistochemistry in the tissue array

Statistics

Distribution between the two treatment arms regarding clinico pathological data and tumour markers were calculated by chi-square-analysis. The relation between VEGF-A VEGFR2 and HER2 and clinico pathological data was calculated by chi-square-analysis and Pearson correlation

For survival analyses all calculations were done according to the intention to treat rule. Recurrence-free survival (RFS) considered distant, local and regional metastasis as primary event as well as breast cancer death, whereas contralateral breast cancer was not included. The Kaplan-Meier method was used to estimate recurrence-free and breast cancer survival and the log-rank test to compare survival in different strata. Cox proportional hazards model was used for estimation of univariate hazard ratios (HRs) and multivariate analysis.

Results

Patient and Tumour Characteristics

Förslag:

Clinical and pathological characteristics according to treatment arm in the original studies and in present studies is given in table 1a for premenopausal patients and 1b for postmenopausal patients, indicating that there was no selection bias in any of the two studies.

The relation betwwen VEGF-A, VEGFR2 and tumor characteristics are given in Table 2a for premenopausal patients and Table 2b for postmenopausal patients.

There was a strong co-expression between VEGF-A and VEGFR2 in both studies. Overexpression of HER 2 was, however, not co-expressed with VEGF-A or VEGFR2, but correlated strongly with NHG 3, ER negativity, PR positivy and decreasing age (table 2a).

TABLE 2
VEGF-A, VEGFR2 and HER2 overexpression in relation to clinico
pathological variables and tumour characteristics

| [symbol] | [symbol] | | [symbol] | | [symbol] | |
|---|---|---|---|---|---|---|
| AGE | 0.08 | (0.07) | −0.04 | (0.4) | −0.01 | (0.03) |
| continuous | | | | | | |
| TUMOR SIZE | 0.03 | (0.5) | −0.01 | (0.8) | 0.07 | (0.2) |
| continuous | | | | | | |
| NODE | 0.02 | (0.6) | 0.001 | (0.98) | 0.08 | (0.1) |
| positivity | | | | | | |
| NHG 3 | 0.1 | (0.04) | 0.14 | (0.003) | 0.2 | (<0.001) |
| ER+ | 0.1 | (0.03) | −0.07 | (0.13) | −0.3 | (<0.001) |
| PR+ | 0.06 | (0.2) | −0.09 | (0.07) | −0.2 | (<0.001) |

| | | | | | |
|---|---|---|---|---|---|
| VEGF-A | — | 0.45 | (<0.001) | −0.03 | (0.5) |

| | | | | |
|---|---|---|---|---|
| VEGFR2 | — | — | −0.04 | (0.4) |

¹ NHG = Nottingham histological grade, ER = estrogen receptor, PR status = progesterone receptor

VEGF-A and VEGFR2 were analyzed as continuous variables and HER2 as categorized (overexpression versus 0-2)

Tamoxifen Response

Premenopausal Patients

ER Status

Recurrence-free survival in 324 ER positive patients was significantly increased by two years of tamoxifen treatment with an absolute risk reduction of 15% from 51% recurrence rate without tamoxifen treatment to 36% recurrence rate in the tamoxifen treated group, whereas OS was not significantly improved by tamoxifen treatment (HR, RFS, 0.6, p=0.01, OS 0.8, p=0.2). In ER negative disease no effect was noted by tamoxifen treatment,(HR, RFS 0.9, p=0.7, OS 0.9, p=0.7).

VEGF-A

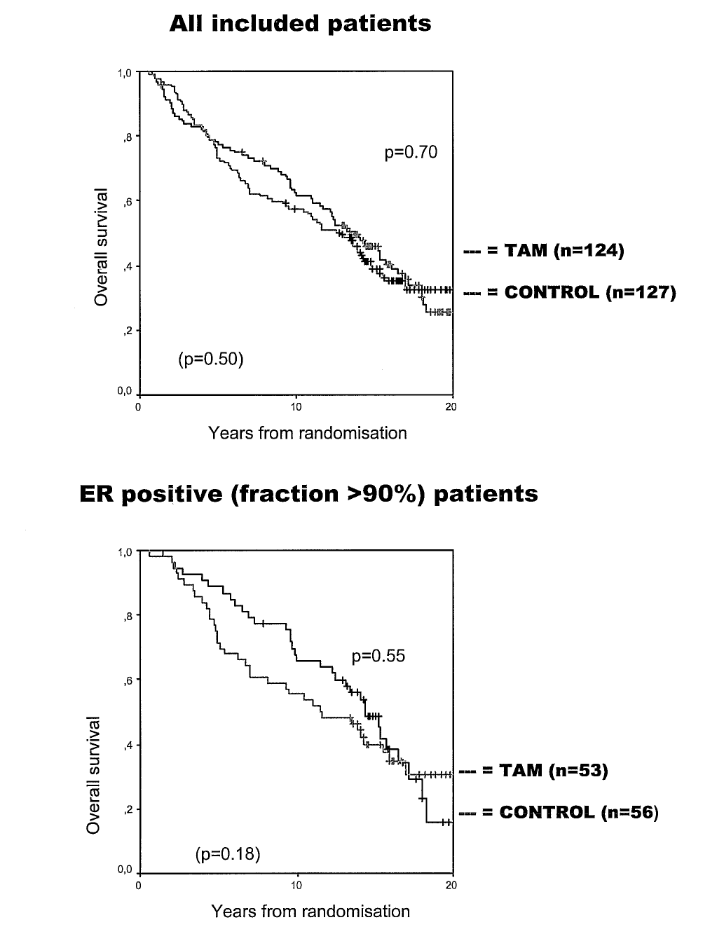

Tamoxifen increased RFS in ER positive disease with VEGF-A grade 0-2 tumors, (HR=0.6 p=0.02, whereas in tumors with VEGF grade 3, no effect was noted, (HR=0.8, p=0.2), FIG. 2. For OS, no difference was observed between VEGFA grade 0-2 and grade 3 tumors by univariate analyses (HR=0.8, p=0.2 and HR=0.8, p=0.8, respectively). This finding was confirmed using a Cox proportional model including a treatment-interaction variable (VEGF (+/−)×TAM (+/−)).

VEGFR2

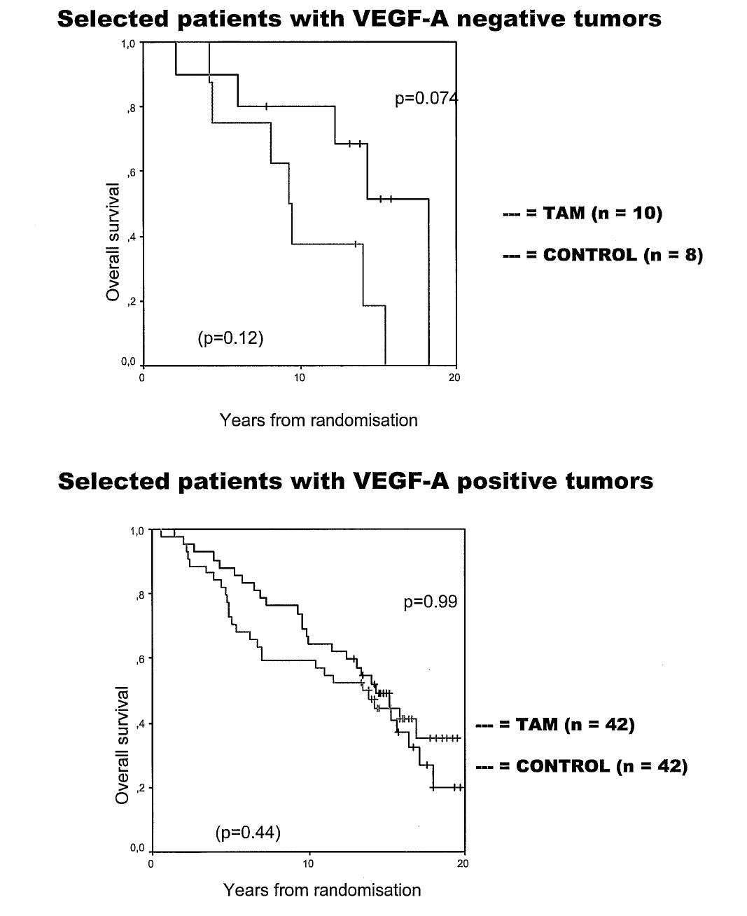

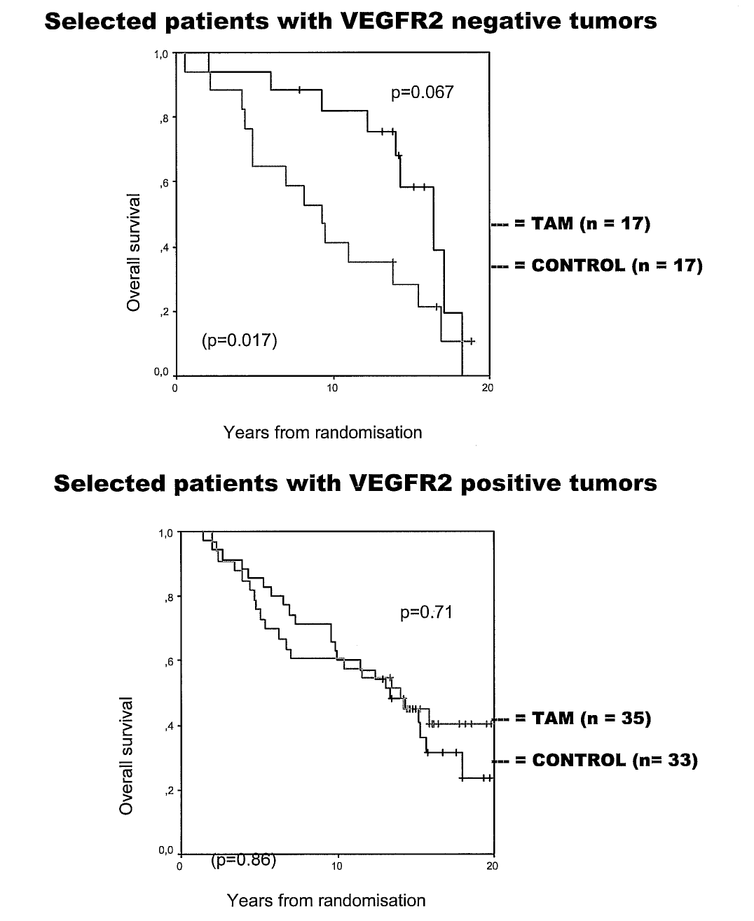

ER positive patients with grade 0, 1 or 2 staining intensity of VEGFR2 was associated with an improved RFS by adjuvant tamoxifen treatment (HR=0.6, p=0.02), whereas patients with tumors with intense expression of VEGFR2 did not benefit from tamoxifen treatment (HR=1.4, p=0.5), as illustrated by Kaplan-Meier estimates, FIG. 3. VEGFR2 grade 0-2 identified a group with increased OS by two years of adjuvant tamoxifen, (HR=0.7, p=0.03), whereas in VEGFR2 3 the tamoxifen effect was approaching a significant adverse effect, (HR=3.4, p=0.07), FIG. 4. The interaction between tamoxifen treatment and VEGFR2 status in ER positive patients is illustrated in Table 4b including a treatment interaction-variable. The interaction-variable between VEGFR2-status and tamoxifen treatment was not significant for RFS, but for OS.

HER2

By univarate analysis including ER positive tumors, RFS was significantly increased by tamoxifen treatment in HER2 low tumors, (HR, 0.6, p=0.03), whereas in tumors with HER2 overexpression, no significant effect by tamoxifen treatment was noted, (HR=0.7, p=0.5). For OS the result by univariate analysis for HER 2 low tumors was HR=0.8, p=0,3 and for HER 2 overexpressing tumor, HR=0.9, p=0.8 The interaction between HER2 status and tamoxifen treatment was further analyzed for both RFS and OS using a multivariate Cox proportional hazards model including an interaction-variable, Table 4.

Postmenopausal Patients

Estrogen Status

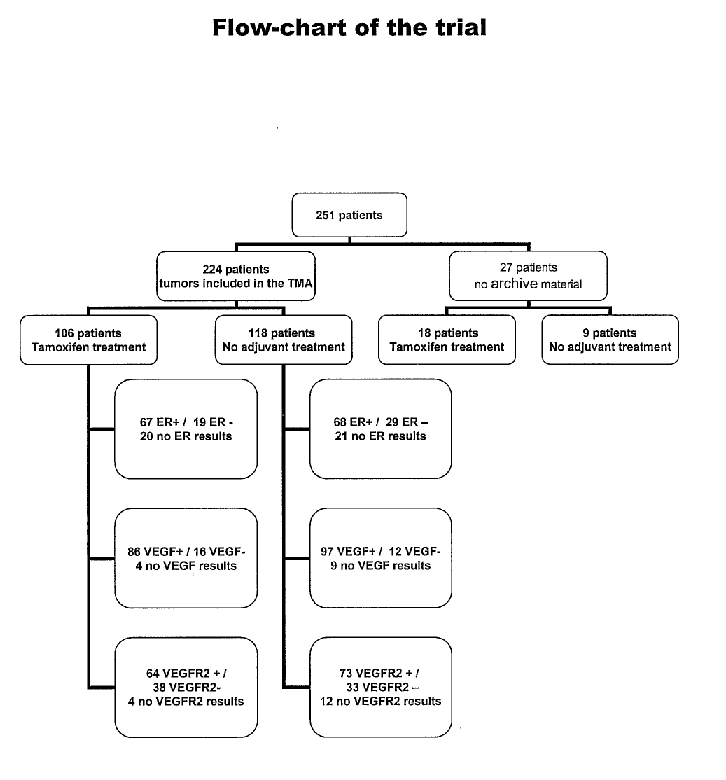

Of the initial 260 patients, survival data was present for 251 and at the last follow-up, 86 patients were alive and 165 deaths were recorded. The median age at death was 75 years (56-96years). The effect on overall survival of tamoxifen treatment for all patients irrespective of hormone receptor content was non-significant, FIG. 1a. The age-distribution in treated vs control group was the same, as well as within analyzed subgroups. For patients with >90% ER postive cells, the tamoxifen response was notable at 10-years, however not strictly significant, FIG. 1b.

VEGF-A and VEGFR2

Patients (n=109) with ER positive tumors with a fraction of stained nuclei >90% were selected for further analysis regarding tamoxifen response. Table 1b demonstrates that there was no selection bias regarding age, node.status and tumor size in the patients selected for further analysis.

The tamoxifen response in terms of OS in VEGF-A negative tumors and positive tumors is given in FIGS. 2a and b, illustrating a notable, but not significant, effect in VEGF negative disease at 10-years (HR 0.3; 95% CI=0.06-1.2, p=0.15), which was not demonstrated for VEGF-A positive tumors (HR 0.8; 95% CI=0.4-1.5, p=0.44). By Cox proportional hazards model including VEGF-status, tamoxifen treatment and an interaction-variable, the interaction-variable for VEGF-A and tamoxifen response was not significant at 10 years, p=0.29.

For VEGFR2 negative tumors, the effect by tamoxifen tratment, was significant at 10 years in terms of OS (HR=0.2; 95% CI=0.06-0.9 p=0.028), contrasting to a non-significant effect in VEGFR2 positive tumors (HR=0.9; 95% CI=0.4-2.0, p=0.9, FIGS. 3a and b. When applying a Cox model for VEGFR2 stautus, tamoxifen treatment and an interaction-variable, the latter indicated a remarkable difference in tamoxifen response between patients with VEGFR2 negative and VEGFR2 positive tumors (HR=0.2, 95% CI: 0.06-1.1, p=0.069), although the p-value was not striclty significant. The results at 10-year DFS was similar, with a p-value for the interaction variable between VEGFR2 status and tamoxifen treatment of 0.055.

Among the two of angiogenic factors, it was nevertheless clear that VEGFR2 was the superior marker for response to tamoxifen treament.

VEGF-A, VEGFR2, HER2 and Prognostic Information

Premenopausal Patients

In untreated patients, VEGF-A grade 3 and VEGFR2 grade 3 yielded no prognostic information by univariate and multivariate analysis including age, tumor size (T2 vs T1), node-status (N1 vs N0), Nottingham histologial grade (NHG) (3 vs 1 and 2), ER status PR status (positive vs negative), and HER 2, for both RFS and OS (Table 3a). However, when stratifying for ER status, VEGFR2 was a significant predictor of favourable prognosis in ER+disease (HR, RFS, 0.4, p=0.02, OS 0.2, p=0.01), and a significant predictor of unfavourable prognosis in ER-disease (HR, RFS 2.3, p=0.02, OS, 3.0, p=0.002), by univariate analysis (Table 3b, c). VEGFR2 was an independent prognostic factor in both ER+ and ER− disease in two multivariate models including the variables listed above (except for hormone receptor content) with. Stratification for ER status did nod add any prognostic information for VEGF-A (table 3 b, c).

HER 2 overexpression was a significant prognostic factor for OS by univariate analysis including all untreated patients and by both univariate and multivariate analysis for RFS and OS in ER+patients (Table 3a, b).

Postmenopausal Patients

VEGF-A and VEGFR2 yielded no prognostic information at 10-year overall survival by cox uni- and multivariate analyses as illustrated in Table 3. Node-status (positive versus negative) was the only independent prognostic factor.

TABLE 3
10-year overall survival with Cox univariate- and
multivariate analysis for 127 untreated patients.

| | UNIVARIATE | | | MULTIVARIATE | | |
|---|---|---|---|---|---|---|
| variable | HR | 95% CI | p-value | HR | 95% CI | p-value |
| Node status | | | | | | |
| N0 | 1.0 | 2.1-6-2 | <0.001 | 3.7 | 1.7-8.3 | 0.001 |
| N+ | 3.6 | | | | | |
| Tumor size | | | | | | |
| T1 | 1.0 | 0.4-1.2 | 0.2 | 1.5 | 0.6-3.3 | 0.4 |
| T2 | 0.7 | | | | | |
| ER status | | | | | | |
| negative | 1.0 | 0.4-1.4 | 0.3 | 0.5 | 0.6-3.6 | 0.5 |
| positive | 0.7 | | | | | |
| PR status | | | | | | |
| negative | 1.0 | 0.3-1.2 | 0.2 | 0.4 | 0.1-1.1 | 0.08 |
| postive | 0.6 | | | | | |
| VEGF-A | | | | | | |
| negative | 1.0 | 0.3-1.6 | 0.4 | 0.6 | 0.2-1.6 | 0.3 |
| postivie | 0.7 | | | | | |
| VEGFR2 | | | | | | |
| negative | 1.0 | 0.4-1.5 | 0.4 | 0.7 | 0.3-1.8 | 0.5 |
| positive | 0.8 | | | | | |

Cox multivariate proportional hazards model for
recurrence-free survival and overall survival in untreated patients

| | RFS | | | OS | | |
|---|---|---|---|---|---|---|
| Variable | HR | 95% CI | p-value | HR | 95% CI | p-value |

A. Estrogen receptor positive patients (n = 173)

| | | | | | | | |
|---|---|---|---|---|---|---|---|
| Age | | 1.0 | 0.9-1.0 | 0.01 | 1.0 | 0.9-1.0 | 0.1 |
| Tumour | T2 v T1 | 1.1 | 0.6-1.8 | 0.8 | 1.1 | 0.6-1.9 | 0.8 |
| size | | | | | | | |
| Node | N+ v N0 | 1.5 | 0.8-2.7 | 0.2 | 2.2 | 1.1-4.3 | 0.03 |
| status | | | | | | | |
| NHG | 3 v 1 + 2 | 1.2 | 0.7-2.0 | 0.4 | 1.6 | 0.9-2.8 | 0.08 |
| VEGFR2 | 3 v 0, 1, 2 | 0.4 | 0.2-1.0 | 0.06 | 0.3 | 0.09-0.9 | 0.03 |
| HER2 | 3 v 0, 1, 2 | 1.6 | 0.8-3.2 | 0.2 | 2.4 | 1.2-4.7 | 0.01 |

B. Estrogen receptor negative patients (n = 72)

| | | | | | | | |
|---|---|---|---|---|---|---|---|
| Age | | 1.0 | 0.9-1.1 | 0.8 | 1.0 | 0.9-1.0 | 0.5 |
| Tumour | T2 v T1 | 2.0 | 0.9-4.3 | 0.08 | 2.2 | 1.0-4.7 | 0.04 |
| size | | | | | | | |
| Node | N+ v N0 | 9.1 | 3.1-26.5 | <0.001 | 8.6 | 3.0-24.8 | <0.001 |
| status | | | | | | | |
| NHG | 3 v 1 + 2 | 1.5 | 0.6-4.0 | 0.4 | 1.2 | 0.4-3.5 | 0.7 |
| VEGFR2 | 3 v 0, 1, 2 | 2.7 | 1.2-6.1 | 0.02 | 4.0 | 1.7-4.2 | 0.001 |
| HER2 | 3 v 0, 1, 2 | 0.6 | 0.4-1.8 | 0.6 | 0.8 | 0.4-1.8 | 0.6 |

Abbreviations:
N0 = node negative, N+ = node positive, ER = estrogen receptor (ER− < 10% fraction of stained nuclei, ER+ ≧ 10% fraction of stained nuclei), PR = progesterone receptor (PR− < 10% fraction of stained nuclei, PR+ ≧ 10% fraction of stained nuclei), VEGF-A = vascular endothelial growth factor-A, VEGFR2 = vascular endothelial growth factor receptor 2.

TABLE 4

| | RFS | | | OS | | |
|---|---|---|---|---|---|---|
| Variable | HR | 95% CI | p-value | HR | 95% CI | p-value |

A. Cox multivariate proportional hazards Model for Interaction of
Tamoxifen Treatment and VEGF-A status in estrogen receptor positive patients

| | | | | | | | |
|---|---|---|---|---|---|---|---|
| Age | | 1.0 | 0.9-1.0 | 0.005 | 1.0 | 0.9-1.0 | 0.8 |
| Tumor size | T2 v T1 | 1.5 | 1.0-2.1 | 0.04 | 1.5 | 0.9-2.6 | 0.1 |
| Node status | N+ v N0 | 2.0 | 1.2-3.2 | 0.005 | 3.1 | 1.8-5.4 | <0.001 |
| NHG | 3 v 1 + 2 | 1.4 | 1.0-1.9 | 0.02 | 1.2 | 0.7-2.0 | 0.5 |
| VEGF-A | 3 + 2 v 0 − 1 | 0.7 | 0.5-1.1 | 0.2 | 1.3 | 0.6-2.5 | 0.4 |
| Randomization | Tam v control | 0.5 | 0.3-0.8 | 0.01 | 1.1 | 0.5-1.1 | 0.8 |
| Interaction- | | 1.5 | 0.8.-3.1 | 0.2 | 0.5 | 0.2-1.4 | 0.2 |
| variable | | | | | | | |

A. Cox Proportional Hazards Model for Interaction of Tamoxifen
Treatment and VEGFR2 status in estrogen receptor positive patients

| | | | | | | | |
|---|---|---|---|---|---|---|---|
| Age | | 1.0 | 0.9-1.0 | 0.007 | 1.0 | 0.9-1.0 | 0.04 |
| Tumor size | T2 v T1 | 1.3 | 0.9-1.9 | 0.1 | 1.2 | 0.9-2.0 | 0.1 |
| Node status | N+ v N0 | 1.7 | 1.1-2.7 | 0.02 | 2.0 | 1.2-3.3 | 0.005 |
| NHG | 3 v 1 + 2 | 1.4 | 1.0-1.9 | 0.04 | 1.7 | 1.2-2.3 | 0.002 |
| VEGFR2 | 3 v 0 − 2 | 0.4 | 0.2-0.9 | 0.02 | 0.2 | 0.07-0.7 | 0.009 |
| Randomization | Tam v control | 0.6 | 0.4-0.8 | 0.002 | 0.6 | 0.4-0.9 | 0.02 |
| Interaction- | | 2.2 | 0.7-7.0 | 0.2 | 5.2 | 1.3-20.6 | 0.02 |
| variable | | | | | | | |

The present studies has provided background data for failing tamoxifen response in pre- and postmenopausal estrogen receptor positive patients in relation to VEGF-A and VEGFR2 in two controlled, randomised trials. HER 2 was additionally analyzed in premenopausal patients. VEGFR-2 has hiterhto not been explored in relation to breast cancer prognosis or treatment prediction, but was in the presented studies the only marker with predictive information.

VEGFR2 overexpression was a strong predictor of favourable clinical outcome in untreated patients with estrogen receptor positive disease. Interestingly, this group of patients did not benefit from adjuvant tamoxifen and a significant interaction was noted between tamoxifen treatment and VEGFR2 status in ER positive patients, even when traditional prognostic markers were taken into account. Although the fraction of patients overexpressing VEGFR2 is limited, the data from this randomised study is worthwhile reporting. VEGFR2 is the most important receptor for VEGF-A and mainly situated on endothelial cells stimulating proliferation and migration via it's tyrosine kinas activity. VEGFR2 has recently been identified on tumour cells both in vivo and in vitro implicating autocrine activity for VEGF-A besides its proangiogenic properties. The intracellular signalling pathways for VEGFR:s in malignant cells seems has therefore been studied and seem to involve both MAPK-activity, ERK 1/2 and p13 in T47D breast cancer cell lines (24). For pancreatic cancer, the ERK pathway has been identified downstream of VEGFR2 resulting in increased proliferative activity (31). The intracellu lar signalling pathway for VEGFR:s in cancer cells can therefore be postulated to, at least in part, be similar to the signalling cascade identified for EGFR and HER2 resulting in a ligand -independent receptor activation of the estrogen receptor (25). VEGFR2 was not associated with HER2 and the observed impaired tamoxifen effect by VEGFR2 overexpression could not be explained by a synchronous overexpression of HER2. Tamoxifen seems to act as an estrogen agonist in tumours overexpressing VEGFR2, although our findings can only be defined as descriptive. Estrogen up regulates VEGF protein levels, and preclinical data has demonstrated that even tamoxifen can stimulate VEGF production. VEGFR2 overexpression therefore seems to identify a small group of patients with favourable prognosis having an impaired and even adverse effect by tamoxifen treatment.

In contrast to the good prognosis for ER positive patients with VEGFR2 overexpression, VEGFR2 overexpression was a strong indicator of relapse and death in ER negative patients in both univariate and multivariate analyses. Different signalling pathways for VEGFR2 could therefore be one explanation to the clinical outcome in ER positive and ER negative tumours. When analysing nuclear fractions of ER status separately (ER 0-10%, 11-50%, 51-75% and >75% nuclear fraction), the hazard ratio for RFS and VEGFR2 overexpression was stepwise decreasing (2.3, 0.8, 0.5, 0.2), further supporting different clinical information by VEGFR2 depending on ER status. VEGF-A, the main ligand for VEGFR2, is correlated to ER negativity and ligand-induced activities associated with VEGF-A, like proliferation and migration, could therefore be more likely to be present in ER negative disease.

This is the first study where the expression of VEGF-A is related to tamoxifen response in a randomised trial. There are several reports about a deteriorated clinical outcome in hormone receptor positive patients with high cytosolic levels of VEGF-A treated with tamoxifen. We found a significant tamoxifen response in tumours with absent and weak expression of tumour specific VEGF-A by univariate analysis, which could not be demonstrated in tumours with intermediate and strong expression of VEGF-A. The result was not consistent in multivariate analysis considering treatment-interaction. The finding is in contrast to the results from non-randomised trials and could possibly be explained by the study design or the selection of patients including only premenopausal patients in addition to methodological explanations. High levels of cytosolic VEGF-A is a prognostic factor for impaired clinical outcome in several independent studies, whereas immunohistochemical determinations of tumour expression of VEGF-A includes only one study with positive results and several without any association with clinical outcome. VEGF-A is produced not only by tumour cells and biological availability of VEGF-A in the extracellular matrix may be better analyzed by a cytosolic based analysis than a tumour cell based method. In a retrospective study analyzed by both methods, cytosolic levels of VEGF-A could discriminate between different prognostic subgroups, whereas tumour specific evaluation of VEGF-A by IHC was not linked to prognostic information (Ryden). This finding was confirmed in the untreated group in this study where VEGF-A had no prognostic information when analyzed by IHC.

HER2 overexpression was in this randomised study linked to impaired tamoxifen response by univariate analysis, but did not remain an independent predictive factor when exploring treatment-interaction. Conflicting results exist regarding HER2 overexpression and tamoxifen response with limited data from randomised studies. In a randomised study of premenopausal patients treated with both oophorectomy and five years of tamoxifen, HER2 status was not associated with treatment response and the beneficial effect of adjuvant treatment in HER2 overexpressing patients was even larger than in HER2 negative patients.

The study cannot clearly explain the role of HER2 status and tamoxifen treatment alone, since an estrogen suppressing therapy was given as well. Additionally, the importance of analysing downstream products of HER2 together with HER2 status when exploring tamoxifen treatment prediction has been addressed. HER2 status should therefore ideally include determination of crucial pathways for ER ligand-independent pathways. The definitive role of HER2 status in tamoxifen response in this randomised study therefore deserves further studies until any definitive conclusion can be drawn. In summary, this randomised study has clearly demonstrated that tumour specific VEGFR2 overexpression may be an interesting marker of impaired tamoxifen response, whereas VEGF-A and HER2 were not linked to prediction of tamoxifen treatment effect. VEGFR2 can theoretically be targeted by VEGFR2 inhibitors in this subgroup of estrogen receptor positive patients and they seem to be relevant in both pre- and postmenopausal breast cancer.

VEGFR2 inhibitors are present of different types, such as monoclonal antibodies and chemical entities, such as N,N-dimethyl glycine ester of a series of 9-alkoxymethyl-12-(3-hydroxypropyl)indeno[2,1-a]pyrrolo[3,4-c]carbazole-5-ones, in particular an optimal substitution with ethoxymethyl 19 (VEGF-R2 IC(50)=4 nM) and isopropoxymethyl 21 (VEGF-R2 IC(50)=8 nM) as highly most potent inhibitors.

Other chemical entities having been provided as potent VEGFR2 inhibitors are:

2-oxo-3-[5-(2-pyrrolidin-1-yl-ethoxy)-1H-indol-2-ylmethylene]-2,3-dihydro-1H-indole-5-sulfonic acid amide,

3-[5-(2-morpholin-4-yl-ethoxy)-1H-indol-2-ylmethylene]-2-oxo-2,3-dihydro-1H-indole-5-sulfonic acid amide,

2-oxo-3-[5-(2-pyrrolidin-1-yl-ethoxy)-1H-indol-2-ylmethylene]-2,3-dihydro-1H-indole-5-sulfonic acid methylamide,

2-oxo-3-[5-(2-pyrrolidin-1-yl-ethoxy)-1H-indol-2-ylmethylene]-2,3-dihydro-1H-indole-5-sulfonic acid dimethylamide,

2-oxo-3-[5-(2-pyrrolidin-1-yl-ethoxy)-1H-indol-2-ylmethylene]-2,3-dihydro-1H-indole-5-sulfonic acid isopropylamide,

2-oxo-3-[5-(2-pyrrolidin-1-yl-ethoxy)-1H-indol-2-ylmethylene]-2,3-dihydro-1H-indole-5-sulfonic acid phenylamide,

5-(2,3-dihydro-indole-1-sulfonyl)-3-[5-(2-pyrrolidin-1-yl-ethoxy)-1H-indol-2-ylmethylene]-35 1,3-dihydro-indol-2-one,

2-oxo-3-[5-(2-pyrrolidin-1-yl-ethoxy)-1H-indol-2-ylmethylene]-2,3-dihydro-1H-indole-5-solfonic acid (3-cloro-phenyl)-amide,

2-oxo-3-[5-(2-pyrrolidin-1-yl-ethoxy)-1H-indol-2-ylmethylene]-2,3-dihydro-1H-indole-5-solfonic acid (3-cloro-phenyl)-methyl-amide,

2-oxo-3-[5-(2-pyrrolidin-1-yl-ethoxy)-1H-indol-2-ylmethylene]-2,3-dihydro-1H-indole-5-solfonic acid (4-cloro-2-flouro-phenyl)-methyl-amide,

5-(3,4-dihydro-2H-quinoline-1-sulfonyl)-3-[S-(2-pyrrolidin-1-yl-ethoxy)-1H-indol-2-ylmethylene]-1,3-dihydro-indol-2-one,

5-(3,4-dihydro-1H-isoquinoline-2-sulfonyl)-3-[S-(2-pyrrolidin-1-yl-ethoxy)-1H-indol-2-ylmethylene]-1,3-dihydro-indol-2-one,

5-(5-bromo-2,3-dihydro-indole-1-sulfonyl)-3-[5-(2-pyrrolidin-1-yl-ethoxy)-1H-indol-2-ylmethylene]-1,3-dihydro-indol-2-one,

3-[5-(2-morpholin-4-yl-ethoxy)-1H-indol-2-ylmethylene]-2-oxo-2,3-dihydro-1H-indol-5-sulfonic acid methylamide,

3-[5-(2-morpholin-4-yl-ethoxy)-1H-indol-2-ylmethylene]-2-oxo-2,3-dihydro-1H-indol-5-sulfonic acid methylamide,

3-[5-(2-morpholin-4-yl-ethoxy)-1H-indol-2-ylmethylene]-2-oxo-2,3-dihydro-1H-indol-5-sulfonic acid isopropylamide,

3-[5-(2-morpholin-4-yl-ethoxy)-1H-indol-2-ylmethylene]-2-oxo-2,3-dihydro-1H-indol-5-sulfonic acid phenylamide,

5-(2,3-dihydro-indole-1-sulfonyl)-3-[S-(2-morpholin-4-yl-ethoxy)-1H-indol-2-ylmethylene]-1,3-dihydro-indol-2-one,

3-[5-(2-morpholin-4-yl-ethoxy)-1H-indol-2-ylmethylene]-2-oxo-2,3-dihydro-1H-indole-5-sulfonic acid (3-cloro-phenyl)-amide,

3-[5-(2-morpholin-4-yl-ethoxy)-1H-indol-2-ylmethylene]-2-oxo-2,3-dihydro-1H-indole-5-sulfonic acid (3-cloro-phenyl)-methyl-amide,

3-[5-(2-morpholin-4-yl-ethoxy)-1H-indol-2-ylmethylene]-2-oxo-2,3-dihydro-1H-indole-5-sulfonic acid (4-cloro-2-flouro-phenyl)-amide,

5-(3,4-dihydro-2H-quinoline-1-sulfonyl)-3-[5-(2-morpholin-4-yl-ethoxy)-1H-indo1-2-ylmethylene]-1,3-dihydro-indol-2-one,

5-(3 ,4-dihydro-H-isoquinoline-2-sulfonyl)-3-[5-(2-morpholin-4-yl-ethoxy)-1H-indol-2-ylmethylene]-1,3-dihydro-indol-2-one,

5-(5-bromo-2,3-dihydro-indole-1-sulfonyi)-3-[5-(2-morpholin-4-yl-ethoxy)-1H-indol-2-ylmethylene]-1,3-dihydro-indol-2-one,

3-(1H-indol-3-ylmethylene)-2-oxo-2,3-dihydro-1H-indole-5-sulfonic acid amide,

3-(2-methyl-1H-indoir3-ylmethylene)-2-oxo-2,3-dihydro-1H-indole-5-sulfonic acid amide,

3-(1H-indol-5-ylmethylene)-2-oxo-2,3-dihydro-1H-indole-5-sulfonic acid amide,

3-(1H-indol-3-ylmethylene)-2-oxo-2,3-dihydro-1H-indole-5-sulfonic acid methylamide,

3-(2-methy[-1H-indol-3-ylmethylene)-2-oxo-2,3-dihydro-1H-indole-5-sulfonic acid methylamide,

3-(1H-indol-5-ylmethylene)-2-oxo-2,3-dihydro-1H-indole-5-sulfonic acid methylamide,

3-(1H-indol-2-ylmethylene)-2-oxo-2,3-dihydro-1H-indole-5-sulfonic acid methylamide,

3-(1H-indol-3-ylmethylene)-2-oxo-2,3-dihydro-1H-indole-5-sulfonic acid dimethylamide,

3-(2-methyl-1H-indol-3-ylmethylene)-2-oxo-2,3-dihydro-1H-indole-5-sulfonic acid dimethylamide,

3-(1H-indol-5-ylmethylene)-2-oxo-2,3-dihydro-1H-indole-5-sulfonic acid dimethylamide,

3-(1H-indol-2-ylmethylene)-2-oxo-2,3-dihydro-1H-indole-5-sulfonic acid dimethylamide,

3-(4-methoxy-1H-indol-2-ylmethylene)-2-oxo-2,3-dihydro-1H-indole-5-sulfonic acid methylamide

1-Piperidin-1-ylmethyl-3-(1-H-pyrrol-2-ylmethylene)-1,3-dihydro-indol-2-one,

1-Morpholin-4-ylmethyl-3-(1H-pyrrol-2-ylmethylene)-1,3-dihydro-indol-2-one,

1-(4-Methyl-piperazin-1-ylmethyl)-3-(1H-pyrrol-2-ylmethylene)-1,3-dihydro-indol-2-one,

1-[(3-Methoxy-propylamino)-methyl]-3-(dH-pyrrol-2-ylmethylene)-1.3-dihydro-indol-2-one,

1-Butylaminomethyl-3-(1H-pyrrol-2-ylmethylene)-1,3-dihydro-indol-2-one,

5-Chloro-3-(3,5-dimethyl-1H-pyrrol-2-ylmethylene)-1-piperidin-1-ylmethyl-1,3-dihydro-indol-2-one,

5-Chloro-3-(3,5-dimethyl-1H-pyrrol-2-ylmethylene)-1-piperidin-1-ylmethyl-1,3-dihydro-indol-2-one,

5-Chloro-3-(3,5-dimethyl-1H-pyrrol-2-ylmethylene)-1-morpholin-4-ylmethyl-1,3-dihydro-indol-2-one,

3-(3H-Isobenzofuran-1-ylidene)-1-piperidin-1-ylmethyl-1,3-dihydro-indol-2-one and

3-(3H-Isobenzofuran-1-ylidene)-1-morpholin-4-ylmethyl-1,3-dihydro-indol-2-o ne.

(Z)-3-{1-[4-(N-(2-aminoethyl)-N-methylsulphonyl-amino)-phenylamino]-phenyl-methylidene}-5-phenylsulphonylamino-2-indolinone,

(Z)-3-{1-[4-(N-(2-dimethylaminoethyl)-N-phenylsulphonyl-amino)-phenylamino)-1-phenyl-methylidene}-5-phenylsulphonylamino-2-indolinone,

(Z)-3-{1-[4-(N-methyl-N-acetyl-amino)-phenylamino]-1-phenyl-methylidene}-5-phenylsulphonylamino-2-indolinone,

(Z)-3-(1-phenylamino-1-phenyl-methylidene)-5-phenylsulphonylamino-2-indolin one,

(Z)-3-[1-(4-chlorophenylamino)-1-phenyl-methylidene]-5-phenylsulphonylamino-2-indolinone,

(Z)-3-{1-[4-(N-(2-propionylamino-ethyl)-N-propionyl-amino)-phenylamino]-1-phenyl-methylidene}-5-phenylsulphonylamino-2-indolinone,

(Z)-3-[1-(4-dimethylaminomethyl-phenylamino)-1-phenyl-methylidene]-5-phenyl-sulphonylamino-2-indole,

(Z)-3-[l-(4-(N-methyl-N-methylsulphonyl-amino)-phenylamino)-1-phenyl-methylidene]-5-phenylsulphonylamino-2-indolinone,

(Z)-3-{1-[4-dimethylaminomethyl-phenylamino]-1-phenyl-methylidene}-5-ethyl-sulphonylamino-2-indolinone,

(Z)-3-{1-[4-(N-benzyl-N-methyl-aminomethyl)-phenylamino]-1-phenyl-methylidene}-5-ethylsulphonylamino-2-indolinone,

(Z)-3-{1-[4-(2-dimethylamino-ethyl)-phenylamino]-1-phenyl-methylidene}-5-ethyl-sulphonylamino-2-indolinone,

(Z)-3-{1-[4-(benzylaminocarbonyl)-phenylamino]-1-phenyl-methylidene}-5-phenyl-sulphonylamino-2-indolinone,

(Z)-3-{1-[4-(N-dimethylaminocarbonylmethyl-N-acetyl-amino)-phenylamino]-1-phenyl-methylidene}-5-phenylsulphonylamino-2-indolinone,

(Z)-3-{1-[4-(N-(2-dimethylamino-ethyl)-N-methylsulphonyl-amino)-phenylamino )-1-phenyl-methylidene}-5-(N-methyl-N-phenylsulphonyl-amino)-2-indolinone,

4-{[(2-Oxo-1,2-dihydro-3H-pyrrolo[3,2-c]pyridin-3-ylidene)methyl]amino]-benzenesulfonamide

4-{[(2 -Oxo-1,2-dihydro-3H-pyrrolo[2,3-c]pyridin-3-ylidene)methyl]amino}-benzenesulfonamide

3-[(1H-Indazol-6-ylamino)methylidene]-1H-pyrrolo[3,2-b]pyridin-2-one

3-[(6-Quinolinylamino)methylidene]-1,3-dihydo-2H-pyrrolo[3,2-b]pyridin-2-one

4-{[(2-Oxo-1,2-dihydro-3H-pyrrolo[2,3-b]pyridin-3-ylidene)methyl]amino}-benzenesulfonamide

3-[(1H-Indazol-6-ylamino)methylidene]-1H-pyrrolo[2,3-b]pyridin-2-one

3-[(6-Quinolinylamino)methylidene]-1,3-dihydo-2H-pyrrolo[2,3-b]pyridin-2-one

4-{[(2-Oxo-5-phenyl-1,2-dihydro-3H-pyrrolo[2,3-b]pyridin-3-ylidene)methyl]amino}-benzenesulfonamide

3-[(1H-Indazol-6-ylamino)methylidene]-5-phenyl-1H-pyrrolo[2,3-b]pyridin-2-one

5-Phenyl-3-[(6-quinolinylamino)methylidene]-1,3-dihydro-2H-pyrrolo[2,3-b]pyridin-2-one

4-({[5-(2-Furyl)-2-oxo-1,2-dihydro-3H-pyrrolo[2,3-b]pyridin-3-ylidene]methyl}amino)-benzenesulfonamide

5-(2-Furyl)-3-[(1H-indazol-6-ylamino)methylidene]-1H-pyrrolo[2,3-b]pyridin-2-one

5-(2-Furyl)-3-[(6-quinolinylamino)methylidene]-1,3-dihydro-2H-pyrrolo[2,3-b]pyridin-2-one

4-({[2-Oxo-5-(3-thienyl)-1,2-dihydro-3H-pyrrolo[2,3-b]pyridin-3-ylidene]methyl}amino)-benzenesulfonamide

3-[(1H-Indazol-6-ylamino)methylidene]-5-(3-thienyl)-1,3-dihydro-2H-pyrrolo[2,3-b]-pyridin-2-one

3-[(6-Quinolinylamino)methylidene]-5-(3-thienyl)-1,3-dihydro-2H-pyrrolo[2,3-b]pyridin-2-one

4-{[(5-Bromo-2-oxo-1,2-dihydro-3H-pyrrolo[2,3-b]pyridin-3-ylidene)methyl]amino}-benzenesulfonamide

5-Bromo-3-[(1H-indazol-6-ylamino)methylidene]-1,3-dihydro-2H-pyrrolo[2,3-b]pyridin-2-one

5-Bromo-3-[(6-quinolinylamino)methylidene]-1,3-dihydro-2H-pyrrolo[2,3-b]pyridin-2-one

4-{[(6-Chloro-2-oxo-1,2-dihydro-3H-pyrrolo[2,3-b]pyridin-3-ylidene)methyl]amino}-benzenesulfonamide

6-Chloro-3-[(1H-indazol-6-ylamino) methylidene]-1,3-dihydro-2H-pyrrolo[2,3-b]pyrid in-2-one

6-Chloro-3-[(6-quinolinylamino)methylidene]-1,3-dihydro-2H-pyrrolo[2,3-b]pyridin-2-one

Ethyl 3-{[4-(aminosulfonyl)anilino]methylidene}2-oxo-1,2-dihydro-3H-pyrrolo[2,3-b]-pyridine-5-carboxylate

Ethyl 3-[(1H-indazol-6-ylamino)methylidene]-2-oxo-1,2-dihydro-3H-pyrrolo[2,3-b]pyridine-5-carboxylate

Ethyl 2-oxo-3-[(6-quinolinylamino)methylidene]-2,3-dihydro-1H-pyrrolo[2,3-b]pyridine-5-carboxylate

4-[N′-(4-Nitro-2-oxo-1,2-dihydro-indol-3-ylidene)-hydrazino]-benzenesulfonamide

4-[N′-(4-Isopropyl-2-oxo-1,2-dihydro-indol-3-ylidene)-hydrazino]-benzenesulfonamide

4-[(4-Hydroxymethyl-2-oxo-1,2-dihydro-indol-3-ylidenemethyl)-amino]-N-methyl-benzenesulfonamide

4-{N′-[2-Oxo-4-(2-pyridin-4-yl-ethyl)-1,2-dihydro-indol-3-ylidene]-hydrazino}-benzenesulfonamide

2-Oxo-3-(4-sulfamoyl-phenylamino-methylene)-2,3-dihydro-1H-indole-4-carboxylic acid ethyl ester

4-[N′-(4-Iodo-2-oxo-1,2-dihydro-indol-3-ylidene)-hydrazino]-benzenesulfonanide

4-[N′-(4-Isobutyl-2-oxo-1,2-dihydro-indol-3-ylidene)-hydrazino]-benzenesulfonamide

4-{N′-[4-(2-Methyl-propenyl)-2-oxo-1,2-dihydro-indol-3-ylidene]-hydrazino}-benzenesulfonamide

4-{N′-[4-(2-Methyl-1-butenyl)-2-oxo-1,2-dihydro-indol-3-ylidene]-hydrazino}-benzenesulfonamide

4-{N′-[4-(2-Methyl-2-butenyl)-2-oxo-1,2-dihydro-indol-3-ylidene]-hydrazino}-benzenesulfonamide

4-{N′-[4-(2-methylbutyl)-2-oxo-1,2-dihydro-indol-3-ylidene]-hydrazino}-benzenesulfonamide

4-[N′-(4-Cyclobutylmethyl-2-oxo-1,2-dihydro-indol-3-ylidene)-hydrazino]-benzenesulfonamide

4-[N′-(4-Cyclobutylidenemethyl-2-oxo-1,2-dihydro-indol-3-ylidene)-hydrazino]-benzenesulfonamide

4-(N′-{4-[2-(4-Hydroxyphenyl)-ethyl]-2-oxo-1,2-dihydro-indol-3-ylidene}-hydrazino)-benznensulfonamide

4-(N′-{4-[2-(4-Hydroxyphenyl)-vinyll]-2-oxo-1,2-dihydro-indol-3-ylidene}-hydrazino)-benzenesulfonamide

4-[N′-(2-Oxo-4-phenoxy-1,2-dihydro-indol-3-ylidene)-hydrazino]-benzenesulfonamide

4-[N′-(4-Isopropoxy-2-oxo-1,2-dihydro-indol-3-ylidene)-hydrazino]-benzenesulfonamide

4-{N′-[2-Oxo-4-(1H-pyrazol-3-yl)-1,2-dihydro-indol-3-ylidene]-hydrazino}-benzene-sulfonamide

4-[(5-Oxazol-5-yl-2-oxo-1,2-dihydro-indol-3-ylidenemethyl)-amino]benzenesulfonamide

2-Oxo-3-[(4-suafamoyl-phenyl)-hydrazone]-2,3-dihydro-1H-indole-5-carboxylic acid

2,3,4,5,6-pentafluorophenyl ester

4-[N′-(5-Nitro-2-oxo-1,2-dihydro-indol-3-ylidene)-hydrazino]-benzenesulfonamide

4-[N′-(5-Hydroxy-2-oxo-1,2-dihydro-indol-3-ylidene)-hydrazino]-benzenesulfonamide

4-[N′-(5-Methyl-2-oxo-1,2-dihydro-indol-3-ylidene)-hydrazino]-benzenesulfon amide;

N-Methyl-4-[N′-(2-oxo-S-[1,2,4]triazol-1-yl-1,2-dihydro-indol-3-ylidene)-hy drazino]-benzenesulfonamide

2-Oxo-3-[(4-sulfamoyl-phenyl)-hydrazono]-2,3-dihydro-1H-indole-5-sulfonic acid sodium salt

3-[(4-Methylsulfamoyl-phenyl)-hydrazono]-2-oxo-2,3-dihydro-1H-indole-5-carboxylic acid amide

2-Oxo-3-(4-sulfamoyl-phenylamino-methylene)-2,3-dihydro-1H-indole-5-carboxylic acid methyl ester

5-Bromo-3-[(4-Methylsulfonyl-phenyl)-hydrazono]-1,3-dihydro-indol-2-one

2-Oxo-3-[(4-sulfamoyl-phenyl)-hydrazono]-2,3-dihydro-1H-indole-5-sulfonic acid amide

4-[N′-(5-Methylsulfonyl-2-oxo-1,2-dihydro-indol-3-ylidene)-hydrazino]-benzenesulfon-amide

3-[(4-Methylsulfamoyl-phenyl)-hydrazono]-2-oxo-2,3-dihydro-1H-indole-5-sulfonic acid methylamide

4-{N′-[5-(1-Hydroxyimino-ethyl)-2-oxo-1,2-dihydro-indol-3-ylidene]-hydrazino}-N-methyl-benzenesulfonamide

4-[1-(5-Oxazol-5-yl-2-oxo-1,2-dihydro-indol-3-ylidene)-ethylamino]-benzenesulfonamide

N,N-Dimethyl-4-[(5-oxazol-5-yl-2-oxo-1,2-dihydro-indol-3-ylidenemethyl)-amino]-benzenesulfonamide

4-[1-(5-Oxazol-5-yl-2-oxo-1,2-dihydro-indol-3-ylidene)-hydrazino]-benzenesulfonamide

4-[(2-Oxo-5-phenyl-1,2-dihydro-indol-3-ylidenemethyl)-amino]-benzenesulfonamide

2-Oxo-3[(4-sulfamoyl-phenyl)-hydrazono]-2,3-dihydro-1H-indole-5-carboxylic acid dimethylamide

2-Oxo-3-[(4-sulfamoyl-phenyl)-hydrazono]-2,3-dihydro-1H-indol-5-carboxylic acid(furan-2-ylmethyl)-amide

2-Oxo-3-[(4-sulfamoyl-phenyl)-hydrazono]-2,3-dihydro-1H-indol-5-carboxylic acid-2,6-dimethoxy-benzylamide

2-Oxo-3[(4-sulfamoyl-phenyl)-hydrazono]-2,3-dihydro-1H-indole-5-carboxylic acid(2-morpholin-4-yl-ethyl)-amide

2-Oxo-3-[(4-sulfamoyl-phenyl)-hydrazono]-2,3-dihydro-1H-indole-5-carboxylic acid(2-imidazol-1-yl-ethyl)-amide

2-Oxo-3-[(4-sulfamoyl-phenyl)-hydrazono]-2,3-dihydro-1H-indole-5-carboxylic acid(3-imidazol-1-yl-propyl)-amide

2-Oxo-3-[(4-sulfamoy-phenyl)-hydrazono]-2,3-dihydro-1H-indole-5-carboxylic acid(2-methoxyethyl)-amide

2-Oxo-3-[(4-sulfamoyl-phenyl)-hydrazono]-2,3-dihydro-1H-indole-5-carboxylic acid(2-hydroxyethyl)-amide

2-Oxo-3-[(4-sulfamoyl-phenyl)-hydrazono]-2,3-dihydro-1H-indole-5-carboxylic acid(3-hydroxypropyl)-amide

2-Oxo-3-[(4-sulfamoyl-phenyl)-hydrazono]-2,3-dihydro-1H-indole-5-carboxylic acid(3-hydroxy-2,2-dimethylpropyl)-amide

2-Oxo-3-[(4-sulfamoyl-phenyl)-hydrazono]-2,3-dihydro-1H-indole-5-carboxylic acid(pyridin-3-ylmethyl)-amide

2-Oxo-3-[(4-sulfamoyl-phenyl)-hydrazono]-2,3-dihydro-1H-indole-5-carboxylic acid(pyridin-4-ylmethyl)-amide

4-[N′-(5-Methoxy-2-oxo-1,2-dihydro-indol-3-ylidene)-hydrazino]-benzenesulfonamide

4-[N′-(5-Amino-2-oxo-1,2-dihydro-indol-3-ylidene)-hydrazino]-benzenesulfonamide hydrochloride

4-[N′-(6-Ethyl-2-oxo-1,2-dihydro-indol-3-ylidene)-hydrazino]-benzenesulfonamide

N-{4-[(2-Oxo-1,2-dihydro-indol-3-ylidenemethyl)-amino]-phenyl}sulfamide

4-[(6-Hydroxymethyl-2-oxo-1,2-dihydro-indol -3-ylidenemethyl) -amino]-benzene sulfonamide

4-[N′-(6-Bromo-2-oxo-1,2-dihydro-indol-3-ylidene) -hydrazino]-benzenesulfonamide

5 4-[N′-(2-Oxo-6-phenoxy-1,2-dihydro-indol-3-ylidene)-hydrazino]-benzenesulfonamide 4-[N′-(6-Ethoxy-2-oxo-1,2-dihydro-indol-3-ylidene)-hydrazino]-benzenesulfonamide

N-Methyl-4-[N′-(4-methyl-5-nitro-2-oxo-1,2-dihydro-indol-3-ylidene)-hydrazino]-benzenesulfonamide

4-[N′-(5,6-Dimethyl -2-oxo-1,2-dihydro-indol-3-ylidene)-hydrazino]-benzenesulfonamide

N-{6-Hydroxy-3-[(4-methylsulfamoylmethyl-phenyl)-hydrazono]-2-oxo-2,3-dihydro-1H-indol-5-yl}-acetamide

4-[N′-(6-Chloro-5-methoxy-2-oxo-1,2-dihydro-indol-3-ylidene)-hydrazino]benzene-sulfonamide

4-[N′-(5-Hydroxy-6-isopropyl-2-oxo-1,2-dihydro-indol-3-ylidene)-hydrazino]-benzenesulfonamide

C-{4-[N′-(4,6-Dichloro-5-methoxy-2-oxo-1,2-dihydro-indol-3-ylidene)-hydrazino]-phenyl}-N-methyl-methanesulfonamide

4-[N′-(4-Chloro-5-hydroxy-6-methyl-2-oxo-1,2-dihydro-indol-3-ylidene)-hydrazino]-benzenesulfonamide

4-[N′-(5-Hydroxy-4,6-dimethyl-2-oxo-1,2-dihydro-indol-3-ylidene)-hydrazino]-benzenesulfonamide

4-[N′-(5-Hydroxy-4,6-dimethyl-2-oxo-1,2-dihydro-indol-3-ylidene)-hydrazino]-phenyl}-N-methyl-methanesulfonamide

2-Oxo-3-(4-sulfamoyl-phenylamino-methylene)-2,3-dihydro-1H-indole-5-carboxylic acid isobutyl ester

3,6-Dihydro[1,2,3]triazolo[4,5-e]indole-7,8-dione8-[N-(4-methoxyphenyl)hydr azone];

3,6-Dihydro[1,2,3]triazolo[4,5-e]indole-7,8-dione8-{N-[4-(1,3-oxazol-5-yl)phenyl]-hydrazone}

3,6-Dihydro[1,2,3]triazolo[4,5-e]indole-7,8-dione8-[N-(4-methylphenyl)hydra zone]

3,6-Dihydro[1,2,3]triazolo[4,5-e]indole-7,8-dione8-(N-{4-[(E)-2-(2-pyridinyl)-ethenyl]phenyl}hydrazone)

6H-[1,3]Thiazolo[5,4-e]indole-7,8-dione8-[N-(3-methoxyphenyl)hydrazone]

5-Hydroxy-4,6-dimethyl-1H-indole-2,3-dione3-[N-(4-methylphenyl)hydrazone]

3,6-Dihydro[1,2,3]triazolo[4,5-e]indole-7,8-dione8-{N-[4-(trifluoromethyl)phenyl]-hydrazone}

6H-[1,3]Thiazolo[5,4-e]indole-7,8-dione8-[N-(3-fluorophenyl)hydrazone]

6H-[1,3]Thiazolo[5,4-e]indole-7,8-dione8-[N-(4-fluorophenyl)hydrazone]

6H-[1,3]Thiazolo[5,4-e]indole-7,8-dione8-[N-(4-bromophenyl)hydrazone]

6-Bromo-3-{[4-(4-morpholinyl)anilino]methylidene}-1,3-dihydro-2H-indol-2-one

6-Bromo-3-{[4-(4-pyridinylmethyl)anilino]methylidene}-1,3-dihydro-2H-indol-2-one

8-[4-Toluidinomethylidene]-6,8-dihydroimidazo[4,5-e]indol-7(3H)-one

8-[(3-Methyl4-nitroanilino)methylidene]-3,6-dihydro[1,2,3]triazolo[4,5-e]indol-7-one

8-{[4-Nitro-3-(trifluoromethyl)anilino]methylidene}-3,6-dihydro[1,2,3]triaz olo[4,5-e]-indol-7-one

8-[(3-Chloro-4-nitroanilino)methylidene]-1,6-dihydro[1,2,3]triazolo[4,5-e]indol-7-one

8-[(3,5-Dimethyl-4-nitroanilino)methylidene]-1,6-dihydro[1,2,3]triazolo[4,5-e]indol-7-one

8-(n4-[2,2,2-Trifluoro-1-hydroxy-1-(trifluoromethyl)ethyl]anilinolmethylidene)-1,6-dihydro[1,2,3]triazolo[4,5-e]indol-7-one

8-{[4-(2-Hydroxyethyl)anilino]methylidene}-1,6-dihydro[1,2,3]triazolo[4,5-e ]indol-7-one

8-{[4-(Methylsulfanyl)anilino]methylidene}-6H-[1,3]thiazolo[5,4-e]indol-7-one

8-[(3,5-Dimethoxyanilino)methylidene]-6H-[1,3]thiazolo[5,4-e]indol-7-one

8-[(4-Hydroxyanilino)methylidene]-6H-[1,3]thiazolo[5,4-e]indol-7-one

1-[(3-Methoxyanilino)methylidene]-1,3-dihydro-2H-pyrrolo[3,2-f]quinolin-2-one

3-{[(2-Oxo-2,3-dihydro-1H-pyrrolo[3,2-f]quinolin-1-ylidene)methyl]amino}benzonitrile

1-[4-Toluidinomethylidene]-1,3-dihydro-2H-pyrrolo[3,2-f]quinolin-2-one

1-[(4-Methoxyanilino)methylidene]-1,3-dihydro-2H-pyrrolo[3,2-f]quinolin-2-one

3-({[7-Oxo-6,7-dihydro[1,2,3]triazolo[4,5-e]indol-8(1H)-ylidene]methyl}amino)-benzonitrile

8-[4-Toluidinomethylidene]-1,6-dihydro[1,2,3]triazolo[4,5-e]indol-7-one

8-[(4-Methoxyanilino)methylidene]-1,6-dihydro[1,2,3]triazolo[4,5-e]indol-7-one

8-{[4-(4-Morpholinyl)anilino]methylidene}-6H-[1,3]thiazolo[5,4-e]indol-7-one

N-(4-{[(7-Oxo-6,7-dihydro-8H-[1,3]thiazolo[5,4-e]indol-8-ylidene)methyl]amino}-phenyl)acetamide

8-{[4-(2-Hydroxyethyl)anilino]methylidene}-6H-[1,3]thiazolo[5,4-e]indol-7-one

8-{[4-(4-Pyridinylmethyl)anilino]methylidene}-6H-[1,3]thiazolo[5,4-e]indol-7-one

4-{(7-Oxo-6,7-dihydro-8H-[1,3]thiazolo[5,4-e]indol-8-ylidene)methyl]amino}benzamide

6-Bromo-3-{[4-(4-morpholinyl)anilino]methylidene}-1,3-dihydro-2H-indol-2-one

4-{[(6-Bromo-2-oxo-1,2-dihydro-3H-indol-3-ylidene)methyl]amino}benzamide

N-(4-{[(6-Bromo-2-oxo-1,2-dihydro-3H-indol-3-ylidene)methyl]amino}phenyl)acetamide

6-Bromo-3-{[4-(2-hydroxyethyl)anilino]methylidene}-1,3-dihydro-2H-indol-2-one

1-{[4-(4-Morpholinyl)anilino]methylidene}-1,3-dihydro-2H-pyrrolo[3,2-f]quinolin-2-one

1-{[4-(4-Pyridinylmethyl)anilino]methylidene}-1,3-dihydro-2H-pyrrolo[3,2-f]quinolin-2-one

N-(4-{[(2-Oxo-2,3-dihydro-1H-pyrrolo[3,2-f]quinolin-1-ylidene)methyl]amino}phenyl)acetamide

1-{[4-(2-Hydroxyethyl)anilino]methylidene}-1,3-dihydro-2H-pyrrolo[3,2-f]quinolin-2-one

4-{[(2-Oxo-2,3-dihydro-H-pyrrolo[3,2-f]quinolin-1-ylidene)methyl]amino}benzamide

1-[(4-HydroxyaniIino)methylidene]-1,3-dihydro-2H-pyrrolo[3,2-f]quinolin-2-one

6-(2-Furyl)-3-{[4-(4-morpholinyl)anilino]methylidene}-1,3-dihydro-2H-indol-2-one

N-[4-({[6-(2-Furyl)-2-oxo-1,2-dihydro-3H-indol-3-ylidene]methyl}amino)phenyl]-acetamide

6-(2-Furyl)-3-{[4-(2 -hydroxyethyl)anilino]methylidene}-1,3-dihydro-2H-indol-2-one

3-{[4-(4-Morpholinyl)anilino]methylidene}-6-vinyl-1,3-dihydro-2H-indo1-2-one

3-{[4-(4-Pyridinylmethyl)anilino]methylidene}-6-vinyl-1,3-dihydro-2H-indol-2-one

N-(4-{[(2-Oxo-6-vinyl-1,2-dihydro-3H-indol-3-ylidene)methyl]amino}phenyl)acetamide

3-{[4-(2-Hydroxyethyl)anilino]methylidene}-6-vinyl-1,3-dihydro-2H-indol-2-one

6-(2-Furyl)-3-{[4-(4-pyridinylmethyl)anilino]methylidene}-1,3-dihydro-2H-indol-2-one

6-(2-Furyl)-3-[(4-hydroxyanilino)methylidene]-1,3-dihydro-2H-indol-2-one

3-{[4-(4-Morpholinyl)anilino]methylidene}-6-(2-thienyl)-1,3-dihydro-2H-indol-2-one

N-[4-(-[2-Oxo-6-(2-thienyl)-1,2-dihydro-3H-indol-3-ylidene]methyl-amino)phenyl]-acetamide

6-Bromo-3-{[3-(hydroxyrethyl)anilino]methylidene}-1,3-dihydro-2H-indol-2-one

6-Bromo-3-{[4-(5-methyl-3-oxo-2,3-dihydro-1H-pyrazol-1-yl)anilino]methylidene}-1,3-dihydro-2H-indol-2-one

3-Ethyl-3-(4-{[(7-oxo-6,7-dihydro-8H-[1,3]thiazolo[5,4-e]indol-8-ylidene)methyl]amino}-phenyl)-2,6-piperidinedione

8-(4-Phenoxyanilino)methylidene]-6H-[1,3]thiazolo[5,4-e]indol-7-one

8-{[4-(Benzyloxy)anilino]methylidene)-6H-[1,3]thiazolo[5,4-e]indol-7-one

Methyl 4-(4-{[(7-oxo-6,7dihydro-8H-f[1,3]thiazolo[5,4-e]indol-8-ylidene)methyl]amino}-phenoxy)benzoate

Methyl 3-(4-{[(7-oxo-6,7-dihydro-8H-[1,3]thiazolo[5,4-e]indol-8-ylidene)methyl]amino}-phenoxy)benzoate

8-{[3-(Hydroxymethyl)anilino]methylidene}-6H-[1,3]thiazolo[5,4-e]indol-7-one

3-{[(7-Oxo-6,7-dihydro-8H-[1,3]thiazolo[5,4-e]indol-8-ylidene)methyl]amino}benzamide

8-{[4-(5-Methyl-3-oxo-2,3-dihydro-1H-pyrazol-1-yl )anilino]methylidene}-6H-[1,3]thiazolo[5,4-e]indol-7-one

Methyl 4-{[(7-Oxo-6,7-dihydro-8H -[1,3]thiazolo[5,4-e]indol-8-ylidene)methyl]amino}benzoate

4-{[(7-Oxo-6,7-dihydro-8H-[1,3]thiazolo[5,4-e]indol-8-ylidene)methyl]amino}benzonitrile

N-Methyl-N-(4-{[(7-oxo-6,7-dihydro-8H-[1,3]thiazolo[5,4-e]indol-8-ylidene)methyl]-amino}phenyl)acetamide

1-[(4-Phenoxyanilino)methylidene]-1,3-dihydro-2H-pyrrolo[3,2-f]quinolin-2-one

1-{[4-(Benzyloxy)anilino]methylidene}-1,3-dihydro-2H-pyrrolo[3,2-f]quinolin-2-one

Methyl 4-(4-{[(2-Oxo-2,3-dihydro-1H-pyrrolo[3,2-f]quinolin-1-ylidene)methyl]amino}-phenoxy)benzoate

Methyl 3-(4-{[(2-Oxo-2,3-dihydro-1H-pyrrolo[3,2-f]quinolin-1-ylidene)methyl]amino}-phenoxy)benzoate

3-Ethyl-3-(4-{[(2-oxo-2,3-dihydro-1H-pyrrolo[3,2-f]quinolin-1-ylidene)methyl]amino}phenyl)-2,6-piperidinedione

1-[(4-Benzoylanilino)methylidene]-1,3-dihydro-2H-pyrrolo[3,2-f]quinolin-2-one

1-{[3-(Hydroxymethyl)anilino]methylidene}-1,3-dihydro-2H-pyrrolo[3,2-f]quinolin-2-one

1-{[4-(5-Methyl-3-oxo-2,3-dihydro-1H-pyrazol-1-yl)anilino]methylidene}-1H-pyrrolo[3,2-f]quinolin-2(3H)-one

1-((E)-{4-[(E)-2-(4-Hydroxyphenyl)ethenyl]anilino}methylidene)-1,3-dihydro-2H-pyrrolo[3,2-f]quinolin-2-one

3-{[(2-Oxo-2,3-dihydro-1H-pyrrolo[3,2-f]quinolin-1-ylidene)methyl]amino}benzamide

4-{[(2-Oxo-2,3-dihydro-1H-pyrrolo[3,2-f]quinolin-1-ylidene)methyl]amino}benzonitrile

Methyl 4-{[(2-oxo-2,3-dihydro-1H-pyrrolo[3,2-f]quinolin-1-ylidene)methyl]amino-benzoate

8-[(4-{[2-(Diethylamino)ethyl]sulfonyl}anilino)methylidene]-6H-[1,3]thiazolo[5,4-e]indol-7-one

1-[(4-{[2-(Diethylamino)ethyl]sulfonyl}anilino)methylidene]-1H-pyrrolo[3,2-f]quinolin-2(3H)-one

3-{[(2-Oxo-6-phenyl-1,2-dihydro-3H-indol-3-ylidene)methyl]amino}benzamide

Diethyl 4-{[(Z and E)-(2-oxo-6-phenyl-1,2-dihydro-3H-indol-3-ylidene)methyl]-amino}benzylphosphonate

3-{[(2-Oxo-6-phenyl-1,2-dihydro-3H-indol-3-ylidene)methyl]amino}benzonitrile

3-{[2-(2-Hydroxyethyl)anilino]methylidene}-6-phenyl-1,3-dihydro-2H-indol-2-one

3-{[3-(Hydroxymethyl)anilino]methylidene}-6-phenyl-1,3-dihydro-2H-indol-2-one

3-[(2-Methoxyanilino)methylidene]-6-phenyl-1,3-dihydro-2H-indol-2-one

3-{[4-(5-Methyl-3-oxo-2,3-dihydro-1H-pyrazol-1-yl)anilino]methylidene}-6-phenyl-1H-indol-2-one.

3-[(4-Iodoanilino)methylidene]-6-phenyl-1H-indol-2-one

3-({[6-(2-Furyl)-2-oxo-1,2-dihydro-3H-indol-3-ylidene]methyl}amino)benzamide

Diethyl 4-({[6-(2-furyl)-2-oxo-1,2-dihydro-3H-indol-3-ylidene]methyl}amino)benzyl-phosphonate

3-({[6-(2-Furyl)-2-oxo-1,2-dihydro-3H-indol-3-ylidene]methyl}amino)benzonitrile

6-(2-Furyl)-3-{[2-(2-hydroxyethyl)anilino]methylidene}-1,3-dihydro-2H-indol-2-one

6-(2-Furyl)-3-{[3-(hydroxymethyl)anilino]methylidene}-1,3-dihydro-2H-indol-2-one

6-(2-Furyl)-3-[(2-methoxyanilino)methylidene]-1,3-dihydro-²H-indol-²-one

6-(2-Furyl)-3-{[4-(5-methyl-3-oxo-2,3-dihydro-1H-pyrazol-1-yl)anilino]methylidene}-1H-indol-2-one

6-(2-Furyl)-3-[(4-iodoanilino)methylidene]-1H-indol-2-one

3,6-Dihydro[1,2,3]triazolo[4,5-e]indole-7,8-dione8-[N-(4-methoxyphenyl)hydrazone]

3,6-Dihydro[1,2,3]triazolo[4,5-e]indole-7,8-dione8-{N-[4-(1,3-oxazol-5-yl)phenyl]-hydrazone}

3,6-Dihydro[1,2,3]triazolo[4,5-e]indole-7,8-dione8-[N-(4-methylphenyl)hydrazone]

3,6-Dihydro[1,2,3]triazolo[4,5-e]indole-7,8-dione8-(N-{4-[(E)-2-(2-pyridinyl)-ethenyl]phenyl}hydrazone)

6H-[1,3]Thiazolo[5,4-e]indole-7,8-dione8-[N-(3-methoxyphenyl)hydrazone]

5-Hydroxy4,6-dimethyl-1H-indole-2,3-dione3-[N-(4-methylphenyl)hydrazone]

3,6-Dihydro[1,2,3]triazolo[4,5-e]indole-7,8-dione8-{N-[4-(trifluoromethyl)phenyl]-hydrazone}

6H-[1,3]Thiazolo[5,4-e]indole-7,8-dione8-[N-(3-fluorophenyl)hydrazone]

6H-[1,3]Thiazolo[5,4-e]indole-7,8-dione8-[N-(4-fluorophenyl)hydrazone]

6H-[1,3]Thiazolo[5,4-e]indole-7,8-dione8-[N-(4-bromophenyl)hydrazone]

3-{[(1-Methyl-3-phenyl-1H-pyrazol-5-yl)amino]methylident}-6-phenyl-1,3-dihydro-2H-indol-2-one

3-{[(4-Hydroxy-1-naphthyl)amino]methylidene}-6-phenyl-1,3-dihydro-2H-indol-2-one

3-{[(1-Ethyl-1H-pyrazol-5-yl)amino]methylidene}-6-phenyl-1,3-dihydro-2H-indol-2-one

3[(1H-Benzimidazol-2-ylamino)methylidene]-6-phenyl-1H-indol-2-one

3-({[6-(4-Morpholinyl)-3-pyridinyl]amino}methylidene)-6-phenyl-1,3-dihydro-2H-indol-2-one

6-(2-Furyl)-3-{[(l-methyl-3-phenyl-1H-pyrazol-5-yl)amino]methylidene}-1,3-dihydro-2H-indol-2-one

6-(2-Furyl)-3-{[(4-hydroxy-1-naphthyl)amino]methylidene}-1,3dihydro-2H-indol-2-one

3-{[(1-Ethyl-1H-pyrazol-5-yl)amino]methylidene}-6-(2-furyl)-1,3-dihydro-2H-indol-2-one

3-[(1H-Benzimidazol-2-ylamino)methylidene]-6-(2-furyl)-1H-indol-2-one

6-(2-Furyl)-3-( [6-(4-morpholinyl)-3-pyridinyl]amino}methylidene)-1,3-dihydro-2H-indol-2-one

and the pharmaceutically acceptable salts, biohydrolyzable esters, biohydrolyzable amides, biohydrolyzable carbamates, solvates, hydrates, affinity reagents or prodrugs thereof in either crystalline or amorphous form.

### REFERENCES

1. Tamoxifen for early breast cancer: an overview of the randomised trials. Early Breast Cancer Trialists' Collaborative Group. Lancet 1998;351(9114):1451-67.

2. Ali S, Coombes R C. Endocrine-responsive breast cancer and strategies for combating resistance. Nat Rev Cancer 2002;2(2):101-12.

3. Bachelder R E, Crago A, Chung J, Wendt M A, Shaw L M, Robinson G, et al. Vascular endothelial growth factor is an autocrine survival factor for neuropilin-expressing breast carcinoma cells. Cancer Res 2001;61(15):5736-40.

4. Bachelder R E, Wendt M A, Mercurio A M. Vascular endothelial growth factor promotes breast carcinoma invasion in an autocrine manner by regulating the chemokine receptor CXCR4. Cancer Res 2002;62(24):7203-6.

5. Benz C C, Scott G K, Sarup I C, Johnson R M, Tripathy D, Coronado E, et al. Estrogen-dependent, tamoxifen-resistant tumourigenic growth of MCF-7 cells transfected with HER2/neu. Breast Cancer Res Treat 1993;24(2):85-95.

6. Berns E M, Klijn J G, Look M P, Grebenchtchikov N, Vossen R, Peters H, et al. Combined vascular endothelial growth factor and TP53 status predicts poor response to tamoxifen therapy in estrogen receptor-positive advanced breast cancer. Clin Cancer Res 2003;9(4):1253-8.

7. Borg A, Baldetorp B, Ferno M, Killander D, Olsson H, Ryden S, et al. ERBB2 amplification is associated with tamoxifen resistance in steroid-receptor positive breast cancer. Cancer Lett 1994;81(2):137-44.

8. Buchler P, Reber H A, Buchler M W, Friess H, Hines O J. VEGF-RII influences the prognosis of pancreatic cancer. Ann Surg 2002;236(6):738-49; discussion 749.

9. Catalano A, Caprari P, Rodilossi S, Betta P, Castellucci M, Casazza A, et al. Cross-talk between vascular endothelial growth factor and semaphorin-3A pathway in the regulation of normal and malignant mesothelial cell proliferation. Faseb 3 2004;18(2):358-60.

10. Coradini D, Biganzoli E, Pellizzaro C, Veneroni S, Oriana S, Ambrogi F, et al. Vascular endothelial growth factor in node-positive breast cancer patients treated with adjuvant tamoxifen. Br J Cancer 2003;89(2):268-70.

11. De Placido S, De Laurentiis M, Carlomagno C, Gallo C, Perrone F, Pepe S, et al. Twenty-year Results of the Naples GUN Randomised Trial: Predictive Factors of Adjuvant Tamoxifen Efficacy in Early Breast Cancer. Clin Cancer Res 2003;9(3):1039-46.

12. Foekens J A, Peters H A, Grebenchtchikov N, Look M P, Meijer-van Gelder M E, Geurts-Moespot A, et al. High tumour levels of vascular endothelial growth factor predict poor response to systemic therapy in advanced breast cancer. Cancer Res 2001;61(14):5407-14.

13. Garvin S, Dabrosin C. Tamoxifen inhibits secretion of vascular endothelial growth factor in breast cancer in vivo. Cancer Res 2003;63(24):8742-8.

14. Gasparini G, Toi M, Miceli R, Vermeulen P B, Dittadi R, Biganzoli E, et al. Clinical relevance of vascular endothelial growth factor and thymidine phosphorylase in patients with node-positive breast cancer treated with either adjuvant chemotherapy or hormone therapy. Cancer J Sci Am 1999;5(2):101-11.

15. Guo P, Fang Q, Tao H Q, Schafer C A, Fenton B M, Ding I, et al. Overexpression of vascular endothelial growth factor by MCF-7 breast cancer cells promotes estrogen-independent tumour growth in vivo. Cancer Res 2003;63(15):4684-91.

16. Hyder S M, Murthy L, Stancel G M. Progestin regulation of vascular endothelial growth factor in human breast cancer cells. Cancer Res 1998;58(3):392-5.

17. Hyder S M, Nawaz Z, Chiappetta C, Stancel G M. Identification of functional estrogen response elements in the gene coding for the potent angiogenic factor vascular endothelial growth factor. Cancer Res 2000;60(12):3183-90.

18. Hyder S M, Stancel G M. Regulation of VEGF in the reproductive tract by sex-steroid hormones. Histol Histopathol 2000;15(1):325-34.

19. Linderholm B, Grankvist K, Wilking N, Johansson M, Tavelin B, Henriksson R. Correlation of vascular endothelial growth factor content with recurrences, survival, and first relapse site in primary node-positive breast carcinoma after adjuvant treatment. J. Clin Oncol 2000;18(7):1423-31.

20. Lissoni P, Fugamalli E, Malugani F, Ardizzoia A, Secondino S, Tancini G, et al. Chemotherapy and angiogenesis in advanced cancer: vascular endothelial growth factor (VEGF) decline as predictor of disease control during taxol therapy in metastatic breast cancer. Int J Biol Markers 2000;15(4):308-11.

21. Love R R, Duc N B, Havighurst T C, Mohsin S K, Zhang Q, DeMets D L, et al. Her-2/neu overexpression and response to oophorectomy plus tamoxifen adjuvant therapy in estrogen receptor-positive premenopausal women with operable breast cancer. J Clin Oncol 30 2003;21(3):453-7.

22. Manders P, Sweep F C, Tjan-Heijnen V C, Geurts-Moespot A, van Tienoven D T, Foekens J A, et al. Vascular endothelial growth factor independently predicts the efficacy of postoperative radiotherapy in node-negative breast cancer patients. Clin Cancer Res 2003;9(17) :6363-70.

23. Miralem T, Steinberg R, Price D, Avraham H. VEGF(165) requires extracellular matrix components to induce mitogenic effects and migratory response in breast cancer cells. Oncogene 2001;20(39):5511-24.

24. Nicholson R I, Hutcheson I R, Knowlden J M, Jones H E, Harper M E, Jordan N, et al. Nonendocrine pathways and endocrine resistance: observations with antiestrogens and signal transduction inhibitors in combination. Clin Cancer Res 2004;10(1 Pt 2):346S-54S.

25. Osborne C K, Bardou V, Hopp T A, Chamness G C, Hilsenbeck S G, Fuqua S A, et al. Role of the estrogen receptor coactivator AIB1 (SRC-3) and HER-2/neu in tamoxifen resistance in breast cancer. J Natl Cancer Inst 2003;95(5):353-61.

26. Ruohola J K, Valve E M, Karkkainen M J, Joukov V, Alitalo K, Harkonen P L. Vascular endothelial growth factors are differentially regulated by steroid hormones and antiestrogens in breast cancer cells. Mol Cell Endocrinol 1999;149(1-2):29-40.

27. Schiff R, Massarweh S A, Shou J, Bharwani L, Mohsin S K, Osborne C K. Cross-talk between estrogen receptor and growth factor pathways as a molecular target for overcoming endocrine resistance. Clin Cancer Res 2004;10(1 Pt 2):331S-6S.

28. Speirs V, Atkin S L. Production of VEGF and expression of the VEGF receptors FIt-1 and KDR in primary cultures of epithelial and stromal cells derived from breast tumours. Br J Cancer 1999;80(5-6):898-903.

29. Stal O, Borg A, Ferno M, Kallstrom A C, Malmstrom P, Nordenskjold B. ErbB2 status and the benefit from two or five years of adjuvant tamoxifen in postmenopausal early stage breast cancer. Ann Oncol 2000;11(12):1545-50.

30. von Marschall Z, Cramer T, Hocker M, Burde R, Plath T, Schirner M, et al. De novo expression of vascular endothelial growth factor in human pancreatic cancer: evidence for an autocrine mitogenic loop. Gastroenterology 2000;119(5):1358-72.

31. Xie B, Tam N N, Tsao S W, Wong Y C. Co-expression of vascular endothelial growth factor (VEGF) and its receptors (flk-1 and flt-1) in hormone-induced mammary cancer in the Noble rat. Br J Cancer 1999;81(8):1335-43.

1. The use of an inhibitor of the VEGFR2 receptor in the preparation of a pharmaceutical preparation for improving tamoxifen treatment response in pre- and postmenopausal breast cancer patients being estrogen receptor positive.

2. The use according to claim 1, in combination with tamoxifen.
